(19) United States
(12) Patent Application Publication
(10) Pub. No.: US 2008/0026047 A1
(43) Pub. Date: Jan. 31, 2008

(54) Cancer Detection Reagents And Uses In Pathology And Diagnostics And Cancer Cell Death

(76) Inventor: Don Adams North, Arlington, TX (US)

Correspondence Address: BAKER BOTTS L.L.P.;PATENT DEPARTMENT, 98 SAN JACINTO BLVD., SUITE 1500, AUSTIN, TX, 78701-4039, US

(21) Appl. No.: 11/690,638

(22) Filed: Mar. 23, 2007

Related U.S. Application Data
(63) Continuation-in-part of application No. 11/339,052, filed on Jan. 24, 2006.
Application No. 11/339,052 is a continuation-in-part of application No. 11/311,594, filed on Dec. 19, 2005.
(60) Provisional application No. 60/646,961, filed on Jan. 25, 2005.
(60) Provisional application No. 60/669,639, filed on Apr. 8, 2005.
(60) Provisional application No. 60/747,260, filed on May 15, 2006.
(60) Provisional application No. 60/743,699, filed on Mar. 23, 2006.

(51) Int. Cl. A61K 9/127; A61K 31/7052; A61P 35/00; C12Q 1/68; C12N 5/08; A61K 39/395

(52) U.S. Cl. 424/450; 424/138.1; 424/155.1; 435/375; 435/6; 514/44

(57) ABSTRACT

Cancer detection reagents have been identified through comparison of cancer mRNAs with the transcriptome of healthy cells. These cancer detection reagents may be used for a variety of purposes including diagnostics and targeted destruction of cancer cells. In diagnostic applications, peripheral blood or other tissue samples from a subject may be analyzed to generate a cancer profile for that subject. That profile may then be used to guide cancer treatment with known agents. It may also be used to generate agents capable of killing the subject's cancer cells in a targeted manner. These agents may include antibodies and siRNA, RNAi, or antisense RNA.

### PRIORITY CLAIM

The present application claims priority as a Continuation-In-Part under 35 U.S.C. §120 to U.S. patent application Ser. No. 11/339,052, filed Jan. 24, 2006, titled “Cancer Markers and Detection Methods,” which claims priority as a continuation-in-part under 35 U.S.C. §120 to U.S. application Ser. No. 11/311,594, filed Dec. 19, 2005, titled “Nucleic Acids for Apoptosis of Cancer Cells,” both of which claim priority under 35 U.S.C. §119(e) to U.S. Provisional Patent Application Ser. No. 60/646,961, filed Jan. 25, 2005, titled “Cancer Detection Reagents and Uses in Pathology and Diagnostics and Targeted Cancer Cell Death” and to U.S. Provisional Patent Application Ser. No. 60/669,639, filed Apr. 8, 2005, titled “Cancer Markers and Detection Methods,” all four of which application are incorporated by reference herein.

The present application further claims priority under 35U.S.C. §119(e) to U.S. Provisional Patent Application Ser. No. 60/743,699, filed Mar. 23, 2006, titled “Cancer Detection Reagents and Uses in Pathology and Diagnosis and Targeted Cancer Cell Death,” and to U.S. Provisional Patent Application Ser. No. 60/747,260, filed May 15, 2006, titled “Cancer Detection Reagents and Uses in Pathology and Diagnosis and Targeted Cancer Cell Death.”

### TECHNICAL FIELD

Embodiments of present invention relate to cancer pathology and therapy, in particular, those pathology-based diagnostics and therapeutics that utilize individualized genetic profiling.

### BACKGROUND

Cancer results when a cell in the body malfunctions and begins to grow uncontrollably. These malfunctions result from mutations in the cell's DNA blueprint. Thus, while early cancer diagnosis focused on the growth properties and the physical appearance of suspected cancer cells, more modern techniques have begun to examine the cell's inner workings.

Not all cancers are caused by the same mutation. Some treatments that work well for particular cancer-causing mutations are ineffective against cancer having other types of mutations and may actually cause more harm than good if inappropriately prescribed. Thus, it is imperative that cancer diagnostics' ability to distinguish different types of cancer keep pace with the ability to treat different types of cancers appropriately. Current diagnostic methods are struggling to match the speed at which new treatments are developed.

Another problem with current cancer diagnostic methods lies in the need for tissue samples to analyze. All presently successful cancer diagnostic methods, other than pure imaging, require cancer cells to be removed from the patient's body. These cells are most commonly obtained from a tissue biopsy. While effective, tissue biopsies are expensive, time-consuming, and painful for the patient. Additionally, the time required to schedule and obtain a tissue biopsy then analyze it causes a delay in treatment and the biopsy process itself may release cancer cells into the blood stream, resulting in increased metastasis.

Even worse, in some cases a tissue biopsy is not possible due to the location of a tumor. In those instances, the exact nature of the cancer cannot be determined until after surgery has been performed and the tumor removed. While these post-operative tests are still useful in directing further treatment of the patient, if the nature of the tumor could be determined in advance, it might be much more feasible to try non-invasive treatments, such as chemotherapy, before putting a patient through the rigors of surgery. Even if surgery were required, the patient might still benefit from a more detailed pre-operative diagnosis. Such a diagnosis might, for example, allow pre-operative treatment with drugs designed to minimize the chance of metastatic spread of cancer cells dislodged from the tumor during surgery. It might also provide greater direction for surgical techniques, such as how much tissue surrounding the tumor to remove.

Currently, some of the most successful cell-based diagnostic methods utilize non-biopsy samples. For example, PAP smears look for cellular irregularities, but utilize cells normally sloughed off by the body. PAP smears continue to save thousands of lives each year by allowing easy and very early detection of cells in the process of becoming cervical cancer.

Because of problems associated with biopsies and the success of simpler methods, such as PAP smears, the medical community has spent years searching for cancer diagnostics using another readily available sample, blood, particularly peripheral blood. Their efforts have met with some success. For example, the progress or recurrence of prostate cancer is readily monitored using a blood test. However, current blood-based cancer diagnostics, like the prostate cancer test, still remain focused on particular types of cancer. The need remains for a cancer diagnostic able to use blood to diagnose a wide variety of cancers or cancer in general.

Outside of tissue-based cancer diagnostics, most diagnostic methods rely on imaging techniques ranging from simple X-rays to MRIs and nuclear imaging, often using cancer- or tissue-targeted contrast agents to produce better images. However, even the most powerful imaging techniques cannot detect tumors smaller than about 2-5 mm in diameter. By the time a tumor has reached that size, it contains thousands of cells. Further, these sophisticated imagining techniques are too expensive to use during early stages of cancer, when the patient otherwise has no symptoms besides a small tumor that could easily be removed. Rather, complicated imaging diagnostics are most often reserved for patients who have had a large primary tumor and are suspected of having developed metastatic cancer. The small tumors detected are actually metastases produced as the cancer has spread. Thus, unlike primary tumors which often contain large numbers of benign cells, the small tumors detected contain thousands of malignant, metastatic cells, each of which is able to seed another tumor elsewhere in the body.

Clearly, detection of small metastatic tumors through current imaging techniques is really a last-ditch effort to save a critically ill patient. If these metastatic cells could be detected much earlier, such as when they first begin to travel through the blood, then a patient could begin receiving treatment for all of the metastatic tumors he or she would likely have while those tumors were still far too small to be detected by diagnostic imaging or any other current techniques. Thus a need exists for much earlier diagnosis of metastatic tumors, or detection of a greatly increased likelihood of metastatic tumors.

Yet another drawback in modern cancer diagnosis relates to its ability to be coupled to treatment. While some common mutations can be diagnosed through tissue samples and used to direct treatment somewhat specific for the patient's type of cancer, this approach is applicable for only a few types of cancer. Currently no diagnostic method is able to detect a wide range of types of cancer or to provide detailed targets for treatment in numerous types of cancer.

Finally, current cancer diagnostics, particularly those that rely upon tissue biopsies, are very poor at monitoring the progress or effectiveness of treatment. Thousands of dollars and possibly even patients' lives could be saved if treating physicians were able to tell when all or a substantial number of the cancer cells, or of a particular type of cancer cell have been eradicated. Additionally, by their nature cancer cells are able to change very rapidly. Thus, they may mutate even further during the course of a treatment, causing what was once a helpful drug to become powerless or harmful. In essence, the cancer cells may become resistant to the drug, much as bacteria become resistant to antibiotics. Cancer treatment would benefit greatly from diagnostic methods able to detect these and other changes that show the effectiveness of treatment or any further mutations of the patient's cancer cells.

Cancer therapies are most often dependent on single mutations, genes and proteins. As the effectiveness of one therapy wears off, another is prescribed. Two shortcomings in this approach are that no one therapy targets enough of the cancer cells to eradicate the disease, and each cancer subject has a unique set of mutations or other abnormalities that prevent any one therapy from addressing the entire population of cancer patients.

It is desirable to have a robust cancer therapy that addresses each cancer subject individually, then exhibits enough potency to kill all or most of the individual's cancer cells without affecting their healthy cells. To accomplish this, one must be able to profile the genetic, mutational individuality of a cancer subject, then quickly react with an individualized drug to attack those cells that fit the identified profile.

### SUMMARY

Embodiments of present invention relate to short isolated nucleic acids at least 17 bases in length having specific sequences, called cancer detection reagents. Cancer detection reagents also include isolated nucleic acids having sequences complementary to any sequence disclosed in this specification. Embodiments of the invention also relate to methods of using these nucleic acids in cancer diagnostics, particularly pathology-based diagnostics. Other methods relate to anti-cancer uses of the cancer detection reagents, such as killing, inducing the body to kill, or hampering the growth of cancer cells, particularly by inducing the targeted death of cancer cells. While in many embodiments more than one cancer detection reagent will be used, in some embodiments the “cancer detection reagents” used may actually be only a single isolated nucleic acid. Further, “subject” as used in the current description may include an entire animal, such as a human, an organ, as tissue, a tumor, blood, a group of cells, or even an individual cell.

Cancer detection reagents, alone or in combination, may be used to determine the cancer profile of a subject. These cancer detection reagents may be used on a variety of samples from the subject, including traditional tumor biopsies as well as peripheral or other blood.

The cancer detection reagents may be used to detect cancer, determine the stage of cancer, or to monitor the process of the cancer or of its treatment. Additionally, testing with the cancer detection reagents may be used to provide a cancer profile showing several mutations or abnormalities present in one or more metastatic cancer cells within the subject. The presence or absence of particular cancer detection reagents or combinations of cancer detection reagents in a subject may indicate the that various therapeutics, including therapeutics including or based on the cancer detection reagents, should or should not be administered to a subject.

Anti-cancer uses of the cancer detection reagents may be selected based on diagnostic results, such as those described above. However, anti-cancer uses may also be used based on other clinical indications that are wholly or partially independent of the detection using the cancer detection reagents. Anti-cancer uses may include antibody treatment based on peptides encoded by the cancer detection reagents, or nucleic acid-based therapies.

Anti-cancer uses may be coupled with diagnostic monitoring to detect the appearance of new cancer detection reagents in a subject or the disappearance of old cancer detection reagents in the subject. Such appearances and disappearance may warrant corresponding changes in the cancer detection reagents or other therapeutics administered to the subject.

Anti-cancer uses may include inducing targeted cancer cell death in a subject. This induction of targeted cell death may occur in vitro or in vivo. Induction of targeted cell death in vitro may be part of a diagnostic method or a combined diagnostic and therapeutic method.

In a whole animal subject, anti-cancer methods may result in reduction of the size of one or more tumors, increased time for other anti-cancer agents to function, or destruction of cancer cells, particularly small numbers of cells such as those that might remain after surgery or form small metastases not detectable using conventional technologies. Even if tumors are not reachable by some treatments, metastatic cells in the blood may be destroyed, hampering future spread of the cancer.

Accordingly, certain embodiments of the invention may fulfill the following objects and present the following advantages:

construction of an individual, genetic cancer profile for a subject's cancer using only subject blood samples;

utilizing an individual, genetic cancer profile as a precursor for the prescription of existing cancer therapies;

utilizing an individual, genetic cancer profile to facilitate the development of new, individualized, cancer cell targeting therapies.

### BRIEF DESCRIPTION OF THE DRAWINGS

The present invention may be better understood through reference to the following detailed description taken in conjunction with the FIGUREs which illustrate various embodiments of the invention.

FIG. 1 illustrates a method of detecting a cancer detection reagent.

FIG. 2 illustrates correlation between individual cancer detection reagents and cancer types.

FIG. 3 illustrates a method for PCR Reduction using cancer detection reagents.

FIG. 4 illustrates a method of blood testing and cancer profiling.

### DETAILED DESCRIPTION

Embodiments of the present invention relate to cancer detection reagents made of short isolated nucleic acids having sequences corresponding to cancer-associated mutations. Cancer detection reagents may include both nucleic acids having a sequence identical to that of the mutant mRNA and the complementary sequence. However, both complementary nucleic acid strands are not required for all aspects of this invention. In some aspects, only one or the other of the complementary nucleic acids will be used. The appropriate nucleic acid strand to use as the specific cancer detection reagent for each application will be apparent to one skilled in the art.

These cancer detection reagents may be used in diagnosis of metastatic cancer, particularly pathology-based diagnosis, including initial diagnosis as well as treatment and disease progression monitoring, and also including monitoring of targeted cancer cell death.

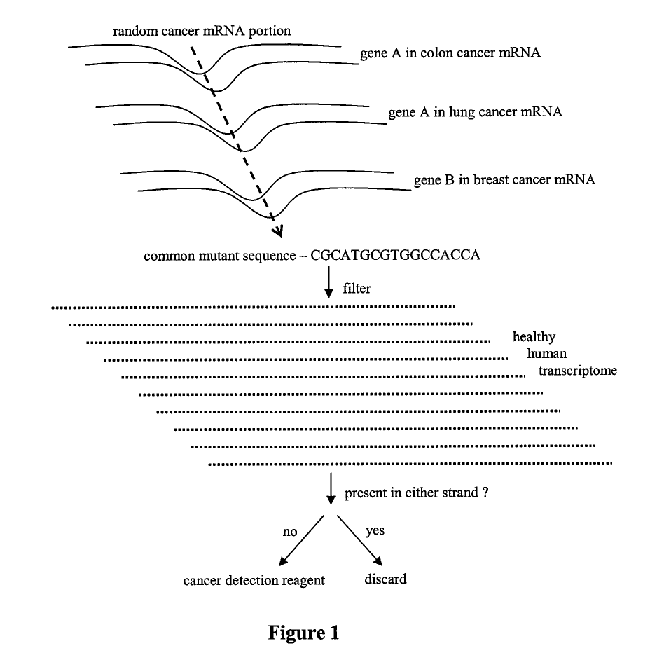

The cancer detection reagent sequences were isolated using proprietary software and information from public databases recording genetic information about cancer and healthy cells and tissues. Specifically, using the proprietary software and supercomputers, random portions of mRNA data from cancer cell lines were compared to all the available mRNA data from all healthy cell lines, as diagramed in FIG. 1.

The resultant database is referred to as the cancer detection reagent sequence hyperset, which contains the sequences of hundreds of thousands of cancer detection reagents, of length 17mer or greater, grouped into supersets according to each cancer type. Each cancer detection reagent in a superset is found at least once in a cancer cell line corresponding to the superset's cancer type. There is redundancy among the supersets because cancer detection reagent sequences appear in supersets for many different cancer types.

The total number of cancer detection reagent sequences in the total cancer hyperset may increase. As new cancer arise, new cancer detection reagents may be created. Based on currently available data, it is known that a superset for a single type of cancer may contain tens of thousands of cancer detection reagents.

The cell lines used to isolate the mRNA molecules that contain the cancer detection reagents were derived from human subjects with cancer. Therefore it is possible to count these cell lines as past occurrences of the cancer detection reagents in humans. This yields a simple method for ranking the likelihood of occurrence of each cancer detection reagent based on its past rate of occurrence in cancer cell lines.

The cancer detection reagents are generally small and thus, unsuitable for genomic mapping. However, the mRNA molecules containing the unisolated cancer detection reagents can be mapped. In this manner, one may determine which genes are associated with each cancer detection reagent.

Many genes may be associated with each cancer detection reagent—the number of genes are normally in direct correlation to the number of unique mRNA molecules containing each cancer detection reagent. Sometimes, hundreds of mRNA molecules contain a cancer detection reagent, yielding many mapped genes. This is evident in TABLE 1.

TABLE 1 lists cancer detection reagents and some associated cancers as well as some other pertinent information, such as PCR annealing temperatures and gene/chromosome location information. These cancer detection reagents may include SNPs, but also include longer mutations suitable for diagnostic and targeted cancer cell death use.

While many of the cancer detection reagent sequences are located in genes with no currently known relevance to cancer, some are located in genes known to be important in cancer. These sequences often represent SNPs, cryptic splicing and other genetic defects.

Cancer detection reagents may be common to many genes and many cancers. This does not mean that every cancer detection reagent will exist in every cancer cell line or cancer subject.

Given the way public data is generated, one would expect much chance and coincidence in any commonality or lack thereof between the cancer detection reagents and cancer cell lines. Each cancer subject is expected to have mRNA containing a subset of cancer detection reagents constituting an individual cancer profile, and identifying which genes may be mutated in that individual. It is possible however, that with a large enough subject pool, the same cancer profile may be observed among different subjects, but nevertheless one does not expect every subject in the pool to have an identical cancer profile.

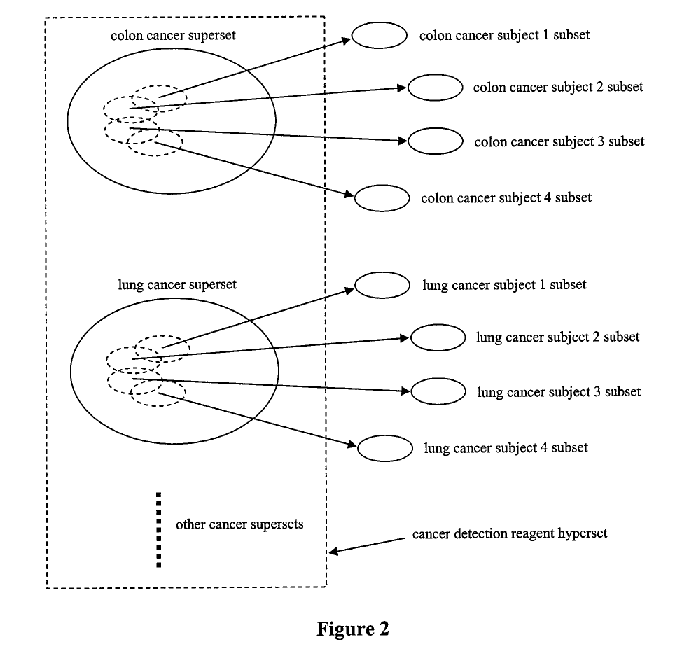

The extent of individualism in cancer is not clearly understood. However, individuality nevertheless appears to correlate with cancer type, as illustrated in FIG. 2. The cancer detection reagent hyperset may constitute all mRNA molecules of length 17mer or greater that are exclusive to cancer cells. Each cancer type then has a corresponding cancer detection reagent superset, and each cancer subject has a cancer detection reagent subset, which is synonymous to their individual cancer profile.

Because TABLE 1 presents a superset of cancer detection reagents, one should not expect to find all of them in a single cancer subject, although this is not impossible. Rather, the detection reagents of TABLE 1 or subcombinations thereof are useful in generating a cancer profile for a particular subject's cancer. By including a large number of cancer detection reagents, a more complete cancer profile may be developed. Additionally, knowledge of what detection reagent sequences are not present in subject's mRNA may also be very useful for diagnosis, including prognosis, as well as cancer progression and treatment monitoring. It may, for example, be useful in selecting a treatment for the subject.

In various embodiments, a cancer profile may be generated using 1 or more, 5 or more, 10 or more, 20 or more, 50 or more, 100 or more, 500 or more, or 1000 or more distinct cancer detection reagents. The cancer detection reagents used in generating the profile may be selected on based on particular criteria, such as overall frequency of occurrence in cancers or previous occurrence in a particular type of cancer.

The cancer detection reagents may be used to test the tumor or blood tissue of actual patients. For example, tests may be performed using PCR, microarray or chip technology. Once the cancer detection reagents are found to be present in the patient, those reagents may be selected to treat the patient or to otherwise guide treatment.

PCR based analyses of samples provides a powerful and rapid means of detecting the presence of cancer detection reagents in a subject's cancer. PCR analyses using amplification primers based on cancer detection reagents enables analysis with small amounts of starting material and enables simultaneous detection of multiple cancer detection reagents. Linear PCR amplification using a single PCR primer based on cancer detection reagents sequences permit quantification of gene expression levels.

Traditional PCR amplifies a set regions of nucleic acid located between the 5′ and 3′ primers. Because both 5′ and 3′ primers are used, the newly created nucleic acid strand becomes available as a template in the next cycle. All primers and PCR conditions are not equally effective at amplification, thus some create new templates at a higher rate than other primers. The effect combined with the ability of new strands to serve as templates results in significant differences in the number of individual nucleic acid strands having the amplified sequence when different primers are used. This difference is related to primer and PCR-condition efficiency rather than the actual number of template strands that were available in the original sample.

A more accurate comparison of the numbers of mRNA molecules containing different cancer detection reagent sequences in a given sample may be obtained using a modified type of PCR herein referred to as “PCR Reduction”. Using this methodology, only 5′ primers are provided. These are able to hybridize with the original template nucleic acid, but not with any strands produced as part of the PCR process because such strands contain sequences identical to, but not complementary to the 5′ primer. Because only the original template nucleic acid may serve as a template for the PCR reaction, differences in copy number of different cancer detection reagent sequences due to primer or PCR efficiency are not so pronounced. Copy number has a much closer correlation with actual number of original templates.

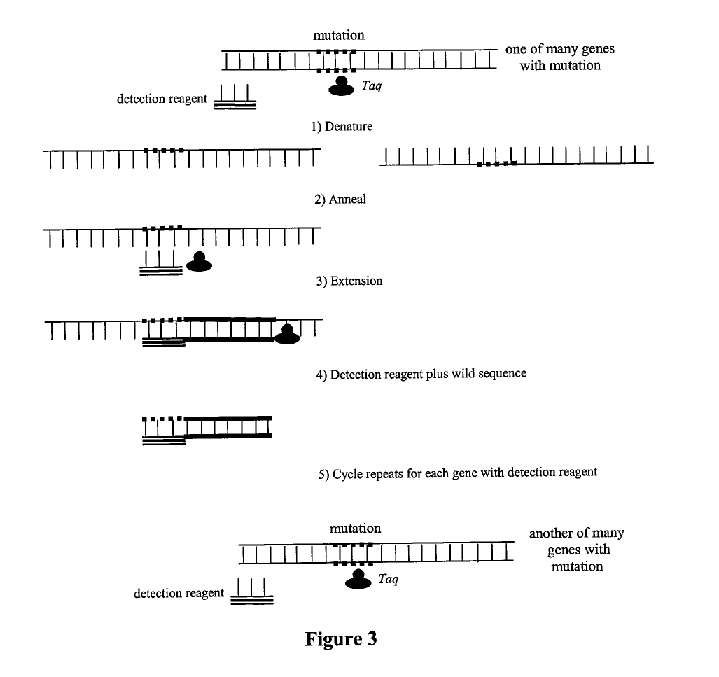

In PCR Reduction, polymerization occurs until the polymerase falls off of the template strand. This tends to leave a trailing end after the 5′ primer. These trailing ends vary somewhat in length, but normally all terminate within several hindered base pairs of the primer. Thus, all of the PCR reaction products may be resolved via electrophoresis on a gel as a single, but slightly blurry band. One example PCR Reduction methodology is illustrated in FIG. 3.

Although amplification of the cancer detection reagents alone might be useful in some embodiments of the invention, in the PCR reduction technique described above the tailing end allows for easy gel-based detection that could not be easily achieved using the small cancer detection reagents alone. If there is no cancer detection reagent sequence present in the sample, then the primers have no template and no band shows up at the expected location after electrophoresis. On the other hand, if the cancer detection reagent sequences are present, a blurry band is present. The intensity of this band may be analyzed using conventional techniques to estimate the relative abundance of templates in the sample containing each detection reagent sequence.

Cancer tissue samples and biopsies usually come from a single tumor, even when multiple tumors are present. In the early stages of cancer most cancer cells are daughters of a parent tumor and often have the same mutations as the cells in the tumor. However, metastatic cancer cells often have different mutations. Further, metastatic tumors, even if initially similar, follow different development pathways and may accumulate different additional mutation over time. Finally, it is well known that many cancer treatments cause further mutations in cancer cells. Therefore, cancer cells in later stages of cancer often do not have the same mutations as those in early stages. Variation in mutations is also often seen among metastatic tumors in the same individual.

Because tumors tend to have individual mutations, it follows that a subject tissue sample taken from a single tumor will likely not contain all the cancer mutations throughout a subject's cancer. A profile of all or most mutations in the subject's body using traditional methodologies thus often requires samples from multiple tumors. In contrast, in embodiments of this invention using blood as a sample, all or most of the mutations may be detected in a single sample because it contains cells from multiple tumors. Further a blood sample may even contain cells from small metastatic tumors not detectable using conventional techniques.

Cancer detection reagents of the present invention may be generally designed to detect mutations that are exclusive to cancer cells, not specific tumors. It has been shown that the cancer detection reagents can detect cancer mutations in cells circulating in the blood. So, one would expect PCR Reduction tests between a tumor tissue sample and a blood sample from the same subject to show an increased number of mutations in the blood. In fact, any cancer profile from a tissue sample alone may be inferior to a blood sample because the tissue cancer profile is actually a profile for the single, biopsied tumor, and not the subject's cancer in general.

Cancer detection reagents represent a special kind of cancer mutation—one that has nucleic acid content exclusive to cancer cells. If such exclusivity were not present, the mutation would not be considered a cancer detection reagent, as shown in FIG. 1. This condition in selecting cancer detection reagents produces reagents that detect useful differences in the genetics of cancer cells. Without such differences, it is difficult or not possible to target the cancer cells while avoiding healthy cells. This is an important criteria for diagnosing and treating cancer.

Cancer profiles may be created for other subjects using a blood sample and the methodologies described herein.

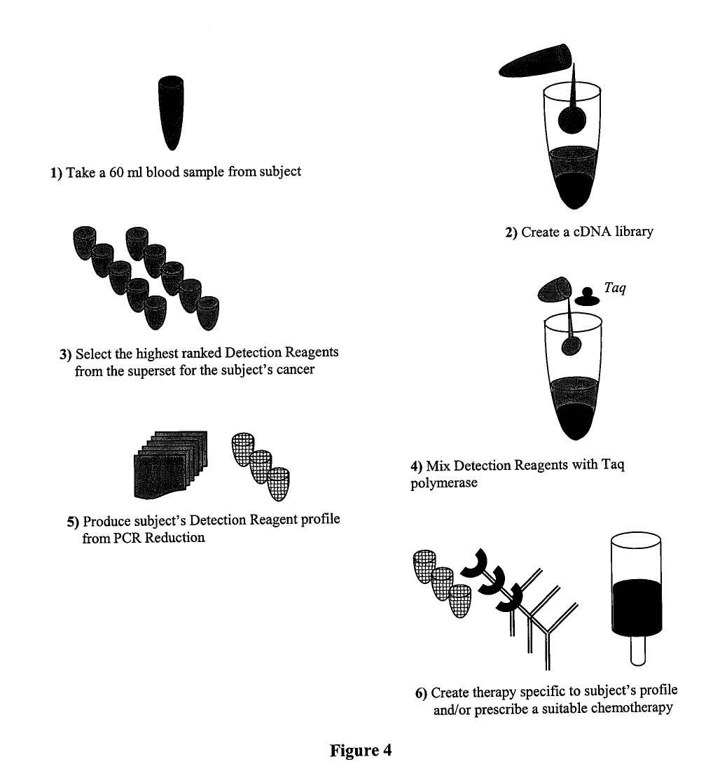

FIG. 4 illustrates steps for one such exemplary methodology. In most instances, a cancer profile may be obtained within a few hours to a few days after obtaining a blood sample from a subject.

Because most cancer detection reagents are associated with a group of genes, one may quickly determine which group of genes are mutating in a subject's cancer in a way that is exclusive to cancer cells. Any subsequent therapy can utilize this genetic information for specific cancer cell targeting. Unfortunately, most existing therapies do not have this kind of targeting capacity. Therefore, the blood tests of the present invention may also be precursory tests for new therapeutics that can use the cancer detection reagents for specific cancer cell targeting.

In a non-limiting embodiment of the invention, the cancer detection reagent may be an antisense oligonucleotide sequence. The antisense sequence may be complementary to at least a portion of the 5′ untranslated, 3′ untranslated or coding sequence of one or several genes in a cancer cell's transcriptome. An oligonucleotide sequence corresponding to the cancer detection reagent may be of sufficient length to specifically interact (hybridize) with the target sequence but not so long that the oligonucleotide is unable to discriminate a single based difference. For example, for specificity the oligonucleotide may be at least six nucleotides in length. Longer sequences can also be used. In a particular embodiment the cancer detection reagent may be 17 nucleotides in length. The maximum length of the sequence will depend on maintaining its hybridization specificity, which depends in turn on the G-C content of the agent, melting temperature (Tm) and other factors, and can be readily determined by calculation or experiment e.g., stringent conditions for detecting hybridization of nucleic acid molecules as set forth in “Current Protocols in Molecular Biology”, Volume I, Ausubel et al., eds. John Wiley: New York N.Y., pp. 2.10.1-2.10.16, first published in 1989 but with annual updating) or by utilization of free software such Osprey (Nucleic Acids Research 32(17):e133) or EMBOSS (http://www.uk.embnet.org/Software/EMBOSS).

Embodiments of the present invention provide for the diagnosis of cancer in subjects including but not limited to humans, domestic pets including but not limited to cats, dogs, hamsters, etc., sport and farm animals including but not limited to horses, cattle, sheep etc. Embodiments of the present invention further provide for utilization of cancer detection reagents in material derived from isolated cells or a sample isolated from a subject in need analysis of disease-state diagnosis. Additional embodiments of the invention include utilization of cancer detection reagents in intact cells or tissues for in situ detection and diagnosis.

Detection of expression of a specific gene under in situ conditions using oligonucleotide probes is well known in the art. Classically oligonucleotide probes have been either 5′ or 3′ end-labeled or 3′ tailed with modified nucleotides that have a “label” attached that can be detected after the probe has hybridized to its target. With end-labeling a single modified ddNTP (that incorporates the label) is added to either the 5′ or the 3′ end of the molecule enzymatically or during probe synthesis. 3′ tailing involves addition of a tail (on average 5-50 nucleotides long of modified dNTPs depending on the method used) using the enzyme terminal transferase (TdT). On the other hand several non-radioactive labels have been used successfully with in situ hybridization include Biotin, digoxin and digoxigenin (DIG), alkaline phosphatase and the fluorescent labels, fluorescein (FITC), Texas Red and rhodamine. The present invention encompasses all the aforementioned methods of probe labeling and detection by in situ hybridization.

A cancer detection reagent may be a DNA or a RNA molecule, or any modification or combination thereof. A cancer detection reagent may contain inter-nucleotide linkages other than phosphodiester bonds, such as phosphorothioate, methylphosphonate, methylphosphodiester, phosphorodithioate, phosphoramidate, phosphotriester, or phosphate ester linkages, resulting in increased stability. Oligonucleotide stability may also be increased by incorporating 3′-deoxythymidine or 2′-substituted nucleotides (substituted with, e.g., alkyl groups) into the oligonucleotides during synthesis or by providing the oligonucleotides as phenylisourea derivatives, or by having other molecules, such as aminoacridine or poly-lysine, linked to the 3′ ends of the oligonucleotides. Modifications of the RNA and/or DNA nucleotides comprising a cancer detection reagent of the invention may be present throughout the oligonucleotide or in selected regions of the oligonucleotide, e.g., the 5′ and/or 3′ ends.

The cancer detection reagents can be made by any method known in the art, including standard chemical synthesis, ligation of constituent oligonucleotides, and transcription of DNA encoding the oligonucleotides.

The present invention in one embodiment provides for the screening of biological tissue that is to be transferred from a member of one animal species to another, including but not limited to one human subject to another. Non-limiting examples of such biological tissue include blood, plasma, skin, bone marrow and major organs such as heart, liver lung, kidney etc. Some embodiments provide for testing a blood or tissue sample for the presence of malignant cells before transfer to another individual. At present many tests are performed on biological material prior to transfusion or transplantation. These tests are performed to ensure pathogen-free material and/or for tissue compatibility. Whether donor tissue contains cells with malignant potential or overtly malignant cells from an undetected cancer has not been of significant concern. The general consensus is that malignant cells from a donor with cancer will not cause transfer of disease to the recipient due to graft rejection. This notion has been recently challenged and there is accumulating evidence of malignancy being transferred between individuals (Dingli and Nowak, 2006 Nature 433:35-36 and references cited therein). The potential for transfer is also enhanced by the use of immunosuppressive drugs in conjunction with organ transplant. Thus, testing biological material for malignant potential is needed. Testing for the presence of malignant cells is limited by the lack of availability of quick and reliable screens which simultaneously test for the presence of multiple markers and that may be performed with a small amount of test material. Embodiments of the present invention provide a way to perform tests for the presence of small amounts of malignant cells in blood or other tissue or organ samples. Testing of candidate donor material may be done by any one of several methods known in the art, non-limiting examples of which are described in the sections above. Methods utilizing cancer detection reagents of the present invention to determine whether donor material contains malignant cells include but are not limited to methods such as PCR, microarray analyses, in situ hybridization etc. In particular embodiments, testing may be used to screen blood in blood banks.

The cancer detection reagents of the present invention may be used for a variety of anti-cancer applications in which they kill cancer cells, induce the body to kill cancer cells, or inhibit growth of cancer cells. These effects may be achieved by the cancer detection reagents along, in combination with one another, or in combination with other therapeutics.

For example, the cancer detection reagents may be used to induce targeted cell death of cancer cells in a variety of ways. First, if they correlate with information known or later developed regarding existing or novel cancer treatments, such as chemotherapy or radiation, they may be used to direct which of these treatments to employ for a subject.

However, the detection reagents of the present invention may also be used to develop novel agents that target the detection reagent nucleic acid sequences or peptides that they encode. These novel agents may be used to induce targeted cancer cell death.

Because the cancer detection reagents of the present invention are absent in the healthy cell transcriptome, they represent cancer-specific targets for inducing cancer cell death.

For example, although some cancer detection reagents may be translated into peptides located primarily within the cell, some are embedded in sequences that normally encode extracellular or membrane proteins. Such sequences are readily known to the art and are considered predictive of the likely cellular location of a protein and portions of it. Accordingly, particularly for proteins with extracellular regions, administration of an antibody specific for a peptide encoded by a cancer detection reagent is expected to induce cell death. Because only cancer cells exhibit these peptides, only cancer cells are targeted and killed by the antibodies.

Antibodies used in conjunction with the present invention may include monoclonal and polyclonal antibodies, non-human, human, and humanized antibodies and any functional fragments thereof.

In addition to protein-based treatments, the cancer detection reagent sequences of the present invention present a particularly promising target for mRNA-based or other nucleic acid-based intervention. Because the cancer detection reagents were isolated through comparison with the transcriptome of a healthy cell, mRNA-based treatments should have no effect on healthy cells because they will lack any mRNA affected by the treatment.

Specifically, mRNA-based treatments may be implemented using nucleic acids including the cancer detection reagent sequences. It is now well known that when mRNA is bound by a complementary nucleic acid within a cell, various cellular mechanisms may then prevent that mRNA from being transcribed into protein. Some of these mechanisms, such as antisense-based mechanisms, appear to rely upon repeated binding of complementary strands to mRNA. Other mechanisms, such as siRNA or RNAi, appear to induce much more long-lasting changes that will prevent translation of the targeted mRNA even from unaltered, single-stranded versions.

In a particular embodiment of the invention, siRNA is created by transcription of plasmid DNA in the cancer cell. The plasmid may be created to allow this transcription activity using a selected cancer detection reagent. A pool of plasmids directed to several cancer detection reagents present in a subject's cancer profile may also be created. After creation of the plasmids, they may be placed in a transfection medium suitable for delivery to cancer cells. It is known that some cells will take up and express bare plasmids. However, a delivery vehicle such as a liposome or deleted viral vector may also be used. Because the plasmids are not expected to have any effect on normal, healthy cells, targeted delivery is not required. It is possible, in some embodiments of the invention, to include additional agents that increase the effectiveness of the delivery vehicle, for example by increasing its diffusion into tissue.

The plasmid as formulated for delivery may then be tested against blood from the cancer subject. Plasmids that function properly should kill cancer cells in the blood in a targeted manner. This activity is readily verifiable though analysis of mRNA in the blood (after a sufficient period of time to allow for degradation of mRNA already produced in the cancer cells before their death, normally less than 1 hour). Lack of damage to normal cells may be verified through visual and other analysis.

Testing in an animal model, such as a primate, may also verify that healthy cells are not affected by the plasmids. Plasmid markers or the cancer detection reagent itself may help verify cellular uptake and/or transcription in vivo.

Plasmids selected for delivery to cancer cells in a subject may then be formulated in a pharmaceutically acceptable manner and administered to the subject. Such administration may include injection into a tumor or injection into the blood stream. Delivery methods may be influenced by the type and location of the cancer as well as other needs of the subject and the formulation of the plasmid agent.

Although a single cancer detection reagent may be used to target multiple genes or gene products in the methods of inducing cancer cell death of the present invention, in some embodiments multiple cancer detection reagents may be targeted to produce an potent effect. Combined agents targeting more than one cancer detection reagent may also be particularly useful if administered to a subject with multiple tumors. The subject's tumors may have differentiated such that every tumor does not contain any one cancer detection reagent sequence. Incorporating agents targeted to multiple cancer detection reagent sequences may allow these differentiated cancer cells to be killed more effectively. Such combined approaches may be particularly powerful against new or small tumors that may not be detected using conventional methods, but nevertheless contain a cancer detection reagent sequence detectable when diagnostic methods of the present invention are used to create a cancer profile.

Thus, targeted cancer cell death may be accomplished using selected methods of the present invention according to a three-step method. First, a cancer profile may be created for the subject. Second, a targeted cancer cell death agent may be created and tested on the subject's blood or other tissue sample. Third, the agent may be administered to the subject to cause targeted death of cancer cells in that subject. This process may be accomplished in as little as three weeks.

Continued monitoring may allow detection of the disappearance of any cancer detection reagents in the subject as well as the appearance of any new ones. The agent or combination of agents administered to the subject may then be changed accordingly.

Table 1 includes cancer detection reagent sequences. Cancer detection reagents may also have sequences complementary to those on Table 1. The cancer detection sequences listed in Table 1 represent reverse complement strands. The sequences listed in Table 1 were selected based on occurrence in cancers overall or rate of occurrence in genes. Incidence (“In.”) indicates the number of times the sequence was located in individual cancers. Each sequence is associated with an identifier corresponding to at least one cancer type in which it has been found. Other types of cancer, the gene or chromosome, and library from which the cancer detection reagent was isolated are also indicated for some sequences. The predicted melting temperature (Tm) is also included for some sequences.

LENGTHY TABLE REFERENCED HERE
US20080026047A1-20080131-T00001
Please refer to the end of the specification for access instructions.

While embodiments of this disclosure have been depicted, described, and are defined by reference to specific example embodiments of the disclosure, such references do not imply a limitation on the disclosure, and no such limitation is to be inferred. The subject matter disclosed is capable of considerable modification, alteration, and equivalents in form and function, as will occur to those ordinarily skilled in the pertinent art and having the benefit of this disclosure. The depicted and described embodiments of this disclosure are examples only, and are not exhaustive of the scope of the disclosure.

LENGTHY TABLE
The patent application contains a lengthy table section. A copy of the table is available in electronic form from the USPTO web site (http://seqdata.uspto.gov/?pageRequest=docDetail&DocID=US20080026047A1). An electronic copy of the table will also be available from the USPTO upon request and payment of the fee set forth in 37 CFR 1.19(b)(3).

1. A method of creating a cancer profile for a subject having or suspected of having cancer comprising:
isolating mRNA from a tissue sample from the subject;
screening the mRNA for at least one cancer detection reagent selected from the group consisting of any oligo from Table 1, and any combination thereof to determine the presence or absence of the cancer detection reagent in the mRNA; and
collating the screening results in a cancer profile indicating the presence or absence of each tested cancer detection reagent in the mRNA.

2. The method of claim 1, wherein the tissue sample comprises peripheral blood.

3. The method of claim 1, further comprising screening the mRNA for at least two cancer detection reagents selected from the group consisting of any oligio from Table 1, and any combination thereof.

4. The method of claim 1, further comprising screening the mRNA for at least 10 cancer detection reagent nucleic acid sequences selected from the group consisting of any oligo from Table 1, and any combination thereof.

5. The method of claim 1, further comprising screening the mRNA for at least 20 cancer detection reagent nucleic acid sequences selected from the group consisting of any oligo from Table 1, and any combination thereof.

6. The method of claim 1, further comprising screening the mRNA for at least 50 cancer detection reagent nucleic acid sequences selected from the group consisting of any oligo from Table 1, and any combination thereof.

7. The method of claim 1, further comprising screening the mRNA for at least 100 cancer detection reagent nucleic acid sequences selected from the group consisting of any oligo from Table 1, and any combination thereof.

8. A method of inducing targeted cancer cell death in a cancer cell comprising administering to the cancer cell at least one antibody targeted to a peptide encoded by a cancer detection reagent nucleic acid sequence selected from the group consisting of any oligo from Table 1, and any combination thereof.

9. The method of claim 8, further comprising administering the antibody to a cancer cell located in a human subject.

10. The method of claim 8, further comprising administering at least two antibodies.

11. The method of claim 8, wherein the antibody comprises a monoclonal antibody.

12. The method of claim 8, wherein the antibody comprises a polyclonal antibody.

13. A method of inducing targeted cancer cell death in a cancer cell comprising administering to the cancer cell at least one siRNA molecule including a cancer detection reagent selected from the group consisting of any oligo from Table 1, and any combination thereof, wherein administering the siRNA molecule results in destruction of cellular mRNA containing the cancer detection reagent.

14. The method of claim 13, further comprising administering the siRNA molecule to a cancer cell in a human subject.

15. The method of claim 13, further comprising administering to the cancer cell a plasmid encoding the siRNA molecule and operable to produce the siRNA molecule in the cancer cell.

16. The method of claim 15, further comprising administering the plasmid in a delivery vehicle.

17. The method of claim 16, wherein the delivery vehicle comprises a liposome.

18. A cancer detection reagent set comprising at least two of any oligo from Table 1.

19. A cancer detection reagent set comprising at least five of any oligo from Table 1.

20. A cancer detection reagent set comprising at least ten of any oligo from Table 1.

21. A cancer detection reagent set comprising at least twenty of any oligo from Table 1.

22. A cancer detection reagent set comprising at least fifty of any oligo from Table 1.

23. A cancer detection reagent set comprising at least one hundred of any oligo from Table 1.

24. A method of detecting a cancer detection reagent in a sample comprising:
contacting the sample with a cancer detection reagent; and determining if nucleic acids containing the cancer detection reagent are present in the sample.

25. The method of claim 24, wherein PCR is performed on the sample using a single primer for a cancer detection reagent to produce a PCR product; and analyzing the PCR product to determine if PCR amplified nucleic acids containing the cancer detection reagent are present in the sample.

26. The method of claim 25, wherein the primer comprises a 5′ primer.

27. The method of claim 24, wherein the sample comprises cDNA.

28. The method of claim 27, further comprising the cDNA derived from a blood or tissue sample.

29. The method of claim 25, wherein analyzing further comprises size-based electrophoretic separation of the PCR product.

30. The method of claim 25, wherein the PCR product comprises a plurality of amplified DNA molecules of multiple lengths, each containing a primer.
